(19) United States
(12) Patent Application Publication
(10) Pub. No.: US 2024/0393320 A1
(43) Pub. Date: Nov. 28, 2024

(54) PLATFORMS FOR DRUG TESTS AGAINST SYNUCLEINOPATHY USING HUMAN BRAIN ORGANOIDS

(71) Applicant: OKINAWA INSTITUTE OF SCIENCE AND TECHNOLOGY SCHOOL CORPORATION, Okinawa (JP)

(72) Inventors: Junghyun JO, Okinawa (JP); Hoang-Dai TRAN, Okinawa (JP); Gordon William ARBUTHNOTT, Okinawa (JP)

(21) Appl. No.: 18/693,722

(22) PCT Filed: Sep. 21, 2022
(86) PCT No.: PCT/JP2022/035166

§ 371 Date: Mar. 20, 2024

(30) Foreign Application Priority Data
Sep. 21, 2021 (JP) 2021-152986

(51) Int. Cl. G01N 33/50; C12N 5/0793

(52) U.S. Cl. CPC G01N 33/5058; C12N 5/0619; G01N 33/5038; C12N 2501/13; C12N 2501/16; C12N 2501/415; C12N 2506/02

(57) ABSTRACT

The present invention provides human striatal-like organoid (hSLO) similar to a huma striatum, a fusion organoid of hSLO and human midbrain-like organoid (hMLO) having functional connections between the hSLO and hMLO, and method of testing drugs using the organoids culture platform.

### TECHINICAL FIELD

The disclosure relates to striatal-like organoids (SLO) and a method of producing the SLOs. The disclosure also relates to a functionally fused striatal-like organoid (SLO) and midbrain-like organoid (MLO). The disclosure also relates to human striatal-like organoids (hSLO) and a method of producing the hSLOs. The disclosure also relates to a functionally fused human striatal-like organoid (hSLO) and human midbrain-like organoid (hMLO).

### BACKGROUND

The striatum is a component of basal ganglia, located in the central human brain, that have a variety of functions including voluntary movement (Hikosaka et al., 2000). It receives and sends out information from and to different brain regions mostly through the projection of specific neurons (Gerfen, 2006; Ingham et al., 1998; Lovinger, 2010; Macpherson et al., 2014). Thus, the striatum is most frequently associated with movement, which is heavily affected by neurodegeneration in patients suffering from Parkinson's disease (PD).

Neural activity between striato-nigral GABAergic neurons in striatum and nigro-striatal dopaminergic (DA) neurons in midbrain motivates the behavior and movement (Albin et al., 1989). Dysfunctions in these neural circuits subsequently contribute to pathogenesis of PD. However, it is poorly understood how these brain regions connect to each other and what causes functional defects in neuronal diseases. Current cell culture and animal models are criticized for their failure to accurately mimic the actual neural circuits in brain. Hence, creating an in vitro brain model to demonstrate and visualize human nigro-striatal pathways will provide a platform for future research on studying neuronal diseases, for example alpha-synuclein (α-syn) pathology.

### BRIEF EXPLANATION OF THE INVENTION

3D organoid model systems are designed to resemble the in vivo organ or tissue from which they were derived. These 3D culture systems can reproduce the complex morphology of differentiated epithelium to enable biologically interaction between cell-cell and cell-matrix. This contrasts with the classical 2D culture models that often share little physical, molecular, or physiological similarity to their tissue of origin. Despite the tremendous promise for in vitro modelling, current brain organoid systems have certain limitations as it cannot reflect every aspect of human brain diseases such as PD.

The generation of human striatal organoids solves several problems:
There has been no stepwise protocol to create reciprocal nigro-striatal and striato-nigral projections between midbrain substantia nigra and striatum where is a particular region of basal ganglia.
There has been no human in vitro neuronal system to demonstrate the synaptic formation between DA neurons and medium spiny neurons (MSNs) in midbrain substantia nigra and striatum, respectively.
There is no system to model α-syn pathology to study the pathological protein propagation between different brain regions.
There is no drug screening/test system based on α-syn pathology as readout using brain organoids culture platform. Most drug screening platforms for neurodegenerative diseases have been formulated by 2D neuronal cell culture systems and animal models which do not recapitulate in vivo human brain milieu and have high risk to misinterpret the result.

In the disclosure, we first describe our method to generate human striatum-like organoid together with characterization data including gene expression analysis, immunohistochemistry analysis and calcium imaging. We next generate fusion organoids using hSLOs and hMLOs, which hMLO has been established by our lab previously, to demonstrate the physical and functional connection between midbrain and striatum with characterization of the reciprocal projections and synaptic formation.

The present disclosure provides, for example, an invention as described below.

(1) A method of culturing an embryoid body (EB) such as a human embryoid body, comprising:
culturing an EB in a first culture medium containing a first factor, for example, for one to seven days, two to six days, or three to five days, wherein the first factor comprises TGF-β and SMAD2/3 signaling pathway inhibitors and/or Wnt inhibitor such as GSK inhibitor (e.g., GSK3 inhibitor or GSK3β inhibitor), preferably TGF-β and SMAD2/3 signal transduction pathway inhibitors and Wnt inhibitor.

(2) The method of (1) above, further comprising:
culturing the obtained EB in a second culture medium containing a third factor, for example, for seven to fourteen days, eight to thirteen days, nine to twelve days, ten to eleven days optionally under orbital shaking conditions,
wherein the third factor comprises a Wnt inhibitor such as GSK inhibitor (e.g., GSK3 inhibitor or GSK3β inhibitor, or XAV939), patterning factors for sonic hedgehog pathway activation such as smoothened agonist and purmorphamine, and activin such as activin A without first factors to obtain an LGE neurospheres.

(3) The method of (2) above, further comprising:
culturing the obtained EB in a third culture medium containing a third factor, wherein the third factor comprises brain-derived neurotrophic factor (BDNF) and/or ascorbic acid without the first and second factors to obtain an organoid such as a human striatal-like organoid.

(4) The method of (3) above, wherein the obtained organoid comprising one or more markers of a mature medium spiny neuron (MSN).

(5) The method of (4) above, wherein the obtained organoid comprising D1 and/or D2 MSN.

(6) The method of (5) above, wherein the obtained organoid expresses at least one or all of interneuron makers such as TH, cholinergic neuron and serotonin neuron markers such as CHAT and 5-HT, glial cell markers such as MBP and GFAP.

(7) The method of any one of (4) to (6) above, wherein the organoid has 1 mm or more in major axis diameter or in diameter, preferably 1 mm to 2 mm in major axis diameter or in diameter.

(8) An isolated organoid comprising: a mature medium spiny neuron (MSN) expressing one or more markers of a mature MSN.

(9) The isolated organoid of (8) above, wherein 50% or more, 60% or more, 70% or more, 80% or more, or 90% or more of the cells contained in the organoid are DARPP32 positive and GABA positive.

(10) The isolated organoid of (8) or (9) above, comprising D1 and/or D2 GABAergic MSN.

(11) The isolated organoid of any one of (8) to (10) above, wherein the organoid expresses at least one or all of interneuron makers such as TH, cholinergic neuron and serotonin neuron markers such as CHAT and 5-HT, glial cell markers such as MBP, S100β, and GFAP.

(12) The isolated organoid of any one of (8) to (11) above, wherein the organoid has 1 mm or more in major axis diameter or in diameter, preferably 1 mm to 2 mm in major axis diameter or in diameter.

(13) A method of producing an organoid, comprising:
providing an organoid of any one of (8) to (12) above (a first organoid), and a second organoid comprising a dopaminergic (DA) neuron, for example, A9-like subtype mDA neuron and A10-like subtype mDA neuron, wherein the organoid preferably expresses one or more of FOXA2, LMXIA, OTX2, DA neuron makers such as TH, DAT, and GIRK2, and
contacting the first organoid and the second organoid to obtain a fusion organoid of the first organoid and the second organoid, wherein a dopaminergic neuron in the second organoid has projection that reaches the first organoid, and a GABAergic MSN in the first organoid has projection that reaches the second organoid.

(14) A fusion organoid of the first organoid of any one of (8) to (12) above and the second organoid comprising a dopaminergic (DA) neuron, for example, A9-like subtype mDA neuron and A10-like subtype mDA neuron, wherein the organoid preferably expresses one or more of FOXA2, LMX1A, OTX2, dopamine neuron makers such as TH, DAT, and GIRK2,
wherein a dopaminergic neuron in the second organoid has projection that reaches the first organoid, and a GABAergic MSN in the first organoid has projection that reaches the second organoid.

(15) The fusion organoid of (14) above, wherein the cells included in the fusion organoid comprises a DA neuron having α-syn aggregation.

(16) A method of testing a candidate drug, comprising:
contacting the candidate drug and the fusion organoid of (14) or (15) above, observing α-synuclein aggregation in a DA neuron, and
selecting the candidate drug that decreases α-synuclein aggregation, compared to a negative control.

(17) The method of (16) above, wherein SNCA is overexpressed in at least a DA neuron or all cells in the second organoid entity or the organoid.

### BRIEF DESCRIPTION OF DRAWINGS

FIG. 1 relates to generation of human striatal-like organoid (hSLOs). (A) Schematic diagram describing the pathways to determine the development of specific human midbrain and striatum. (B) Schematic diagram illustrating the strategy to generate hSLOs. (C) DIC images illustrate the typical morphology of cells at day 10, 20, 30 and 60 of the differentiation and the quantification of EBs in diameter in (D). Scale bar=500 μm. Error bars represent mean±SEM (n=8).

FIG. 2 shows the results of qRT-PCR showing the expression pluripotency and early lateral ganglionic eminence (LGE) markers in the early time of the differentiation. Quantitative RT-PCR analysis of cells dissociated from hSLOs for NANOG, OCT4, ASCL1, DLX5, GSX2 and EBF1. Error bars represent mean±SEM (n=3).

FIG. 3 shows immunohistochemistry analysis to show the differentiation efficiency of GABAergic neurons at day 60. (A) Cryosection of an hSLO at day 60 immunostained for CTIP2 and GABA. Scale bar=20 μm. (B) Quantification of A. Error bars represent mean±SEM (n=3).

FIG. 4 shows Immunohistochemistry analysis to show the expression of functional GABAergic marker DARPP32 and GABA and quantification, white scale bar=50 μm. Error bars represent mean±SEM (n=3).

FIG. 5 shows immunohistochemistry analysis to show the presence of subtypes GABAergic neurons in hSLO. (A) Cryosection of an hSLO immunostained for GABA and Subtance-P. (B) Cryosection of an hSLO immunostained for GABA and DRD2. Scale bar=5 μm.

FIG. 6 shows the result of immunohistochemistry analysis to show the expression of different cell types in hSLOs, similar with human striatum. Cryosection of an hSLO at day 60 and 90 immunostained for (A) MAP2, TH and DAPI, (B) CHAT, 5′HT and DAPI, (C) MBP and DAPI, (D) GFAP and DAPI. Scale bars=50 μm.

FIG. 7 shows the result of the characterization of dissociated neurons in hSLOs. (A) Representative image showing dissociated hSLOs cells labeled with the AAVmDlx::EGFP reporter, neurons are immunostained for EGFP and CTIP2. (B) Representative image showing dissociated hSLOs cells labeled with the AAV-mDIx::EGFP reporter, zoom in panel showing (B′) growth cone and (B″) dendritic spines with arrows showing the location of growth cones and dendritic spines. White scale bar=20 μm, yellow scale bar=5 μm.

FIG. 8 shows the result of calcium imaging and the tracing peaks showing the calcium activity of hSLO. The spiking of 9 single neurons in hSLO at day 150 were extracted on the right panel. Scale bar=200 μm.

FIG. 9 shows the result of immunohistochemistry analysis to demonstrate the reciprocal projection of neuron between hSLOs and hMLOs. (A) A simplified schematic diagram demonstrating the projection of neurons between striatum and substantia nigra in basal ganglia. (B) 3D immunostaining of clearing hMLO-hSLO fusion organoid with hSLO organoid expressing AAV-hSyn1::EGFP and hMLO without labeling. (C) 3D immunostaining of clearing hMLO-hSLO fusion organoid with hMLO organoid carrying TH-EGFP DA neurons reporter and hSLO without labeling. Scale bar=200 μm.

FIG. 10 shows the result of immunohistochemistry analysis to demonstrate the synaptic formation of neurons in hMLO-hSLO fusion organoid. (A) Fusion organoid generated from H9 TH-EGFP hMLO and H9 hSLO immunostained for EGFP, PSD95 and SYN1 showing the expression of pre- and post synaptic markers along side with the projected neurons from hMLO. (B) Fusion organoid generated from H9 TH-EGFP hMLO and H9 hSLO immunostained for EGFP, VMAT2 and GABA showing the expression of VMAT2 and GABA along side with the projected neurons from hMLO. (C) Fusion organoid generated from H9 hMLO and H9-AAV-hSyn1::EGFP hSLO immunostained for EGFP, PSD95 and SYN1 showing the expression of pre—and post synaptic markers along side with the projected neurons from hSLO. (B) Fusion organoid generated from H9 hMLO and H9-AAV-hSyn1::EGFP hSLO immunostained for EGFP, VGAT and GABA showing the expression of VGAT and GABA along side with the projected neurons from hSLO. Red scale bars=1 μm. White scale bars=10 μm.

FIG. 11 shows the result of α-syn pathogenesis modeling in hMLO-hSLO fusion organoids. (A). Whole mount staining images of W/T and SNCA O/E fusion organoid stained for EGFP showing the projection of TH-EGFP⁺ neurons from hMLO to hSLO at 21 d.p.f. and quantification of (mean±SEM; *p<0.05, n=4). White scale bar=200 μm, yellow scale bar=50 μm. (B) Lentiviral construct to generate SNCAlinker-mKO2 overexpression cell line. (C) Whole mount images of fusion organoids stained for mKO2 antibody to label the SNCA-linker-mKO2 propagation from hSLO to hMLO and vise versa, White scale bar=100 μm, yellow scale bar=50 μm. (D) quantification of C (mean±SEM; ****p<0.0001, n=4).

FIG. 12 shows a model of anti-PD drug test using fusion organoids.

### DETAILED DESCRIPTION

The term “embryoid body” (EB) as used herein refers to a three-dimensional aggregate of pluripotent cells or preferably pluripotent stem cells (PSCs). EBs are formed by pluripotent cells such as embryonic stem cells (ESCs) through the intramolecular binding of the Ca²⁺ dependent adhesion molecule E-cadherin expressed on the pluripotent cells. When cultured as single cells in the absence of antidifferentiation factors, PSCs spontaneously aggregate to form EBs. Such spontaneous formation is often accomplished in bulk suspension cultures whereby the dish is coated with non-adhesive materials, such as agar or hydrophilic polymers, to promote the preferential adhesion between single cells, rather than to the culture substrate.

The term “embryonic stem cells” as used herein refers to a pluripotent stem cell that can be derived from the inner cell mass of a blastocyst from animals, for example, mammals such as rodents including mouse and rat, primates including human and monkeys.

The term “pluripotent stem cell” as used herein refers to a stem cell having a pluripotency. Pluripotency is a potential of cells to differentiate into any of the three germ layers, including endoderm, mesoderm and ectoderm, but not into extraembryonic tissues like the placenta. A pluripotent stem cell can be artificially induced from a non-pluripotent cell such as an adult somatic cell, by inducing a forced expression of a combination of certain reprogramming factors. For example, a forced expression of Oct4, Sox2, Klf4 and c-Myc can generate an induced pluripotent stem cell (iPS cell) from a fibroblast and the like.

The term “organoid” refers to a cell aggregate that can be cultured in vitro and usually contains one or more types of cells to form a three-dimensional structure. Some of organoids have a similar tissue structure to an original organ in a body. Organoids having a similar function to an organ can have a therapeutic effect to a disease caused by a reduced function of the organ. Organoids having a similar structure to an organ were generated to study a development of the organ.

The disclosure provides a method of culturing an embryoid body (EB). The EB is preferably an animal EB, more preferably a mammal EB, still more preferably a human EB. The EB can be obtained by culturing pluripotent stem cells such as ES cells or iPS cells as explained above.

In an embodiment, the method comprises culturing an EB in a first culture medium containing a first factor, for example, for one to seven days, two to six days, or three to five days. The first factor may comprise a TGF-β signaling pathway inhibitor and/or a Wnt inhibitor, preferably a TGF-β signaling pathway inhibitor and a Wnt inhibitor such as GSK inhibitor (e.g., GSK3 inhibitor or GSK3β inhibitor).

Examples of TGF-β signaling pathway inhibitors include, for example, but not limited to, (i) one or more selected from the group consisting of ALK4 inhibitor, ALK5 inhibitor, ALK7 inhibitor, SMAD inhibitor such as SMAD2/3 inhibitor including SMAD2/3 phosphorylation inhibitor, and a multiple inhibitor (e.g., a dual inhibitor) for two or more selected from the group consisting of ALK4, ALK5, ALK7, SMAD such as SMAD2/3 and SMAD2/3 phosphorylation preferably a dual inhibitor for TGF-β and SMAD2/3; (ii) one or more selected from the group consisting of LY364947, SB-525334, SD-208, and SB-505124; 616452 and 616453; GW788388 and GW6604; LY580276, which are disclosed in WO2015/002724A, which is herein incorporated by reference in its entirety; or (iii) SB-431542. In an embodiment, Examples of TGF-β signaling pathway inhibitors includes pan-TGF-beta/Smad Inhibitors such as LDN-193189 and K02288; and selective TGF-beta/Smad Inhibitors such as SB431542 and Galunisertib. Dorsomorphin can also be uses as a TGF-β signaling pathway inhibitor.

Examples of Wnt inhibitors include, for example, but not limited to Adavivint (SM04690), IM-12, Lanatoside C, M435-1279, Wnt-C59 (C59), Atranorin, Box5, Isoquercitrin, AZD2858, CCT251545, PNU-74654, IWP-2, CP21R7 (CP21), IWR1-endo, Ginsenoside Rh4, FIDAS-3, Gigantol, AZ6102, IWR-1-exo, Stenoparib (E7449), Indirubin-3′-oxime, Capmatinib (INCB28060), WAY-316606, iCRT3, FH535, IWP-O1, LF3, Prodigiosin, KY19382 (A3051), WIKI4, Heparan Sulfate, Fosconvivint (ICG-001), Triptonide, XAV-939, IWP-4, LGK-974, Foxy-5, MSAB, Laduviglusib (CHIR-99021) HCl, KY-05009, KY1220, IQ-1, KYA1797K, Harminc, G244-LM, KY02111, JW55, PH-064, and Laduviglusib (CHIR-99021).

In a preferable embodiment, the first factor comprises SB431542, XAV-939, and Dorsomorphin.

In an embodiment, the method further comprises culturing the obtained EB in a second culture medium containing a second factor, for example, for a suitable period, for example, for seven to fourteen days, eight to thirteen days, nine to twelve days, or ten to eleven days. The second factor may comprise a patterning factors for sonic hedgehog pathway activation such as smoothened receptor agonists, for example, smoothened agonist (SAG) and purmorphamine. The culturing can be done in the presence of Activin A. In a preferable embodiment, the second factor comprises XAV939, Activin A, SAG, and purmorphamine. This culturing is preferably performed without the first factors. This culturing process may allow the EB to modulate the differentiation towards lateral ganglionic eminence (LGE), which give rise to the striatum during development.

In an embodiment, the method further comprises culturing the obtained EB in a fourth culture medium containing a third factor to obtain an organoid such as a striatal-like organoid (e.g., hSLO). The third factor may comprise brain-derived neurotrophic factor (BDNF) and/or ascorbic acid. This culturing is preferably performed without the first and second factors.

In an embodiment, the striatal-like organoid (e.g., hSLO) preferably has 500 μm or more, 600 μm or more, 700 μm or more, 800 μm or more, 900 μm or more, 1000 μm or more, 1100 μm or more, 1200 μm or more, or 1300 μm or more in diameter. The striatal-like organoid (e.g., hSLO) comprises both of D1 and D2 subtypes GABAergic medium spiny neurons (MSNs) such as D1 GABAergic MSNs expressing dopamine receptor D1 (DRD1) and Substance-P, and D2 GABAergic MSNs expressing DRD2 and enkephalin. In an embodiment, the striatal-like organoid (e.g., hSLO) may express one or more markers for lateral ganglionic eminence (LGE) (LGE markers) such as ASCL1, DLX2, GSX2 and EBF1. In an embodiment, the striatal-like organoid (e.g., hSLO) may further expresses one or more early neuroectodermal markers such as SOX1, and SOX2. In an embodiment, the striatal-like organoid (e.g., hSLO) preferably expresses one or more markers of a mature medium spiny neuron (MSN) such as DARPP32. In an embodiment, the striatal-like organoid (e.g., hSLO) preferably expresses one or more markers selected from the group consisting of interneuron makers such as TH, cholinergic neuron and serotonin neuron markers such as CHAT and 5-HT, glial cell markers such as MBP and GFAP. In a preferable embodiment, at least 30%, 35%, 40%, 45%, 50%, 55%, or 60% of the striatal-like organoid (e.g., hSLO) may be GABAergic neurons. In an embodiment, the GABAergic neurons express GABA and COUP-TF-interacting protein 2 (CTIP2). In a preferable embodiment, the striatal-like organoid (e.g., hSLO) may have one or more axonal branches and growth cones at the axon terminus as well as the formation of dendritic spines. The disclosure provides any of these striatal-like organoid (e.g., hSLO).

The disclosure provides a method of producing an organoid. The method may comprise providing a first organoid and a second organoid. The first organoid is a striatal-like organoid (e.g., hSLO), and the second organoid comprises a dopaminergic neuron (DA), for example, a midbrain dopaminergic (mDA) neuron. In a preferable embodiment, the mDA neuron is selected from the group consisting of A9-like subtype mDA neuron and A10-like subtype mDA neuron. In a preferable embodiment, the second organoid preferably expresses one or more of FOXA2, LMXIA, OTX2, dopamine neuron makers such as TH, DAT, and GIRK2. In a preferable embodiment, the second organoid is midbrain-like organoid (MLO), more preferably human MLO (hMLO). In a preferable embodiment, the organoid can be obtained by contacting and fusing a striatal-like organoid (SLO) with a midbrain-like organoid (MLO).

In all of the embodiments, SLO is preferably hSLO and MLO is preferably hMLO.

In an embodiment, the organoid or fused SLO and MLO comprises projections from SLO to MLO. In an embodiment, the organoid or fused SLO and MLO comprises projections from MLO to SLO. In a preferable embodiment, the organoid or fused SLO and MLO comprises reciprocal projections between SLO and MLO. In a preferable embodiment, the projections may form a neuronal circuit, more preferably an electro-physiologically functional neuronal circuit.

In an embodiment, neurons, preferably a mDNA neurons, in the MLO expresses α-synuclein (α-syn) and preferably has α-syn aggregation. In this embodiment, the neuron, preferably a mDNA neuron, may comprise a gene encoding α-syn (SNCA) operably linked to a control sequence such as a promoter (e.g., PoIII promoter). The disclosure provides the organoid or fused SLO and MLO, wherein the neuron in MLO expresses α-synuclein (α-syn), preferably wherein the organoid or fused SLO and MLO has a detectable α-syn aggregation.

The disclosure provides a method of testing a candidate drug or screening a candidate drug for Parkinson's disease (PD) therapy. The method comprises providing the organoid or fused SLO and MLO. The method comprises contacting the candidate drug with the organoid or fused SLO and MLO, and then, observing α-synuclein (particularly, the presence or absence of α-synuclein aggregation or the degree of α-synuclein aggregation) in the organoid or fused SLO and MLO (preferably mDA neurons). The method further comprises selecting the candidate drug that decreases α-synuclein aggregation, compared to a negative control such as a vehicle-treated group.

In all of the embodiments, culturing is performed under suitable conditions in a suitable culture medium. In an embodiment, the culture medium may be a serum-free medium. In an embodiment, the culture medium may be a chemically defined medium, where all of the chemicals used are known. Examples of culture medium that can be used herein include, but not limited to Eagle's minimal essential medium (EMEM), alpha minimum essential medium (aMEM), Dulbecco's modified Eagle's medium (DMEM), Dulbecco's Modified Eagle medium/Nutrient Mixture F-12 (DMEM/F-12) Roswell Park Memorial Institute medium (RPMI or RPMI 1640), Glasgow's Minimal Essential Medium (GMEM), Biggers, Gwatkin, and Judah medium (BGJ), Biggers, Gwatkin, and Judah medium Fitton-Jackson modification (BGJb), Basal Medium Eagle (BME), Brinster's medium for ovum culture (BMOC-3), Connaught Medical Research Laboratories medium (CMRL), neurobasal medium, CO₂-Independent medium, Ham's F-10 Nutrient Mixture, Ham's F-12 Nutrient Mixture, Improved MEM, Iscove's modified Dulbecco's medium (IMDM), medium 199, Leibovitz's L-15, McCoy's 5A, MCDB 131, Media 199, mTeSR media, Minimum Essential Media (MEM), Modified Eagle Medium (MEM), Waymouth's MB 752/1, Williams' Media E, or combinations, known substitutions or modifications thereof. Minimal medium typically contains a carbon source such as glucose; salts; essential elements such as magnesium, nitrogen, phosphorus, and sulphur; and water. Any cell culture media may be supplemented with further components, as and when required based on the experiment to be performed, the cell type in questions, as well as the required status of the cell. Cell culture supplements are, but are not limited to, serum, amino acids (e.g., L-glutamine), chemical compounds, salts, buffering salts or agents, antibiotics, antimycotics, cytokines, growth factors, hormones, lipids, and derivatives thereof. The culture can typically be performed under 5% CO₂ conditions at 37° C.

In all of the embodiments, a suitable amount of each of the first, second, and third factors is contained in each of the medium.

### Example 1

Establishment of the Protocol and Characterization of hSLOs

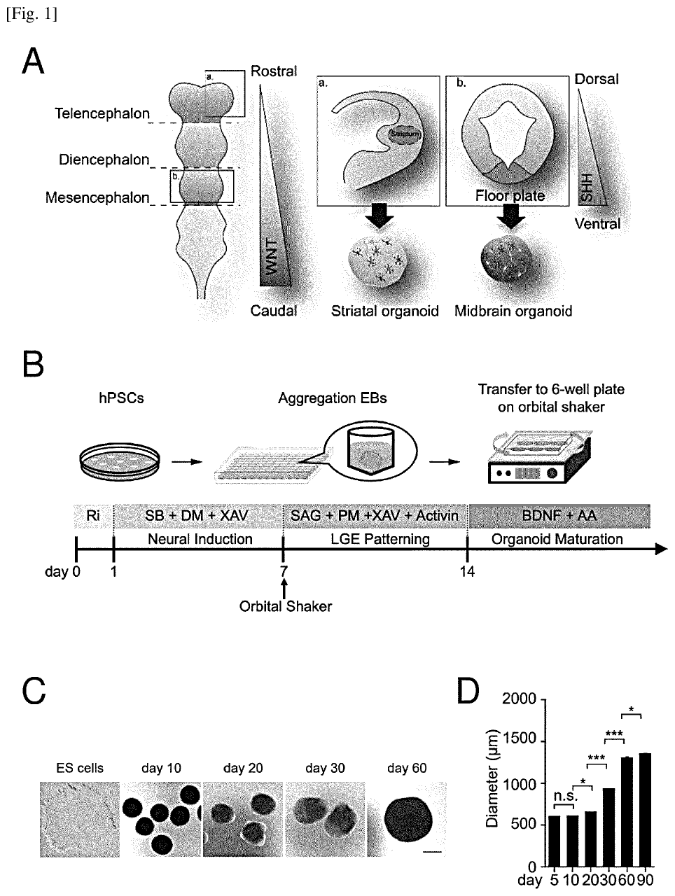

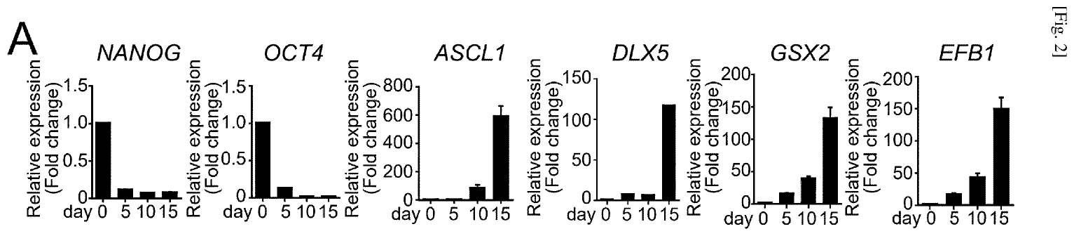

Similar to an approach we previously used to generate hMLOs (Jo et al., 2016), we applied several small molecules to promote neuroectodermal differentiation toward dorsal striatum (FIG. 1A). First, hESC were dissociated to generate single cells and 10,000 cells were plated in V-bottom 96 well plates to form embryo bodies (EBs). At day 1, these EBs were culture in the neuro-induction medium containing DMEM/F12 (Nacalai): Neurobasal (Gibco) (1:1), 1:100 N2 supplement (Invitrogen), 1:50 B27 without vitamin A (Invitrogen), 1% GlutaMAX (Invitrogen), 1% minimum essential media-nonessential amino acid (Invitrogen), 0.1% β-mercaptocthanol (Invitrogen) supplemented with dual-SMAD inhibitor (SB431542, 10 μM, Stemolecule and Dorsomorphin, 2 M, Sigma-Aldrich)) together with WNT inhibitor (XAV939, 0.8 UM, StemCell Technologies). At day 7, medium was supplemented with patterning factors including SAG (0.5 μM, StemCell Technologies) and Purmorphamine (0.5 μM, Stemolecule) to modulate the differentiation towards lateral ganglionic eminence (LGE), which give rise to the striatum during development (FIG. 1B). Importantly, we include Activin A (50 ng/ml, Gibco) to promote the differentiation of striatal neurons through LGE patterning, as reported previously (Arber et al., 2015). EBs were transferred to an orbital shaker from day 7. At day 14, the patterning factors was removed, and organoids were maintained in neuronal media supplemented with BDNF (Peprotech, 10 ng/ml) and Ascorbic Acid (Sigma-Aldrich, 100 μM) (FIG. 1B). By tracing the development across multiple batches, these organoids grew up to 1.3 mm in diameter up to day 60 of the differentiation (FIG. 1C). The investigation of organoid development by qRT-PCR analysis showed decreased expression of pluripotency markers such as NANOG, OCT4, and robust expression of early LGE markers such as ASCL1, DLX5, GSX2 and EBF1 (FIG. 2).

It is known that human striatum consists of over 90% GABAergic MSNs. MSNs can be divided into two half subpopulations based on their axonal projection capacity and neurochemical content: dopamine receptor D1 (DRD1)—and Substance-P-expressing MSNs whereas DRD2—and enkephalin-expressing MSNs (Graveland and DiFiglia, 1985). Previous studies have shown that D1 MSNs send output to the internal globus pallidus (GPi) and the substantia nigra pars reticulata (SNr), forming direct striato-nigral pathway in basal ganglia. On the other hand, striatum receives dopaminergic input from the substantia nigra pars compacta (SNpc) in nigro-striatal pathway in which dopaminergic neurons from SNpc project to striatum and release dopamine from its axon terminal to influence GABAergic MSNs located in the striatum (Yager et al., 2015). This neural circuit also plays an important role in movement. Dysfunction of the nigro-striatal and striato-nigral pathway has been documented as the cause of multiple neuronal diseases such as PD (Goto et al., 1989).

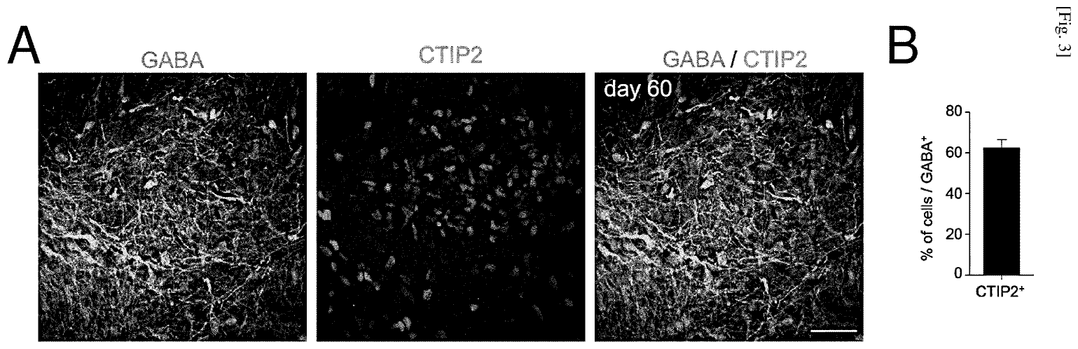

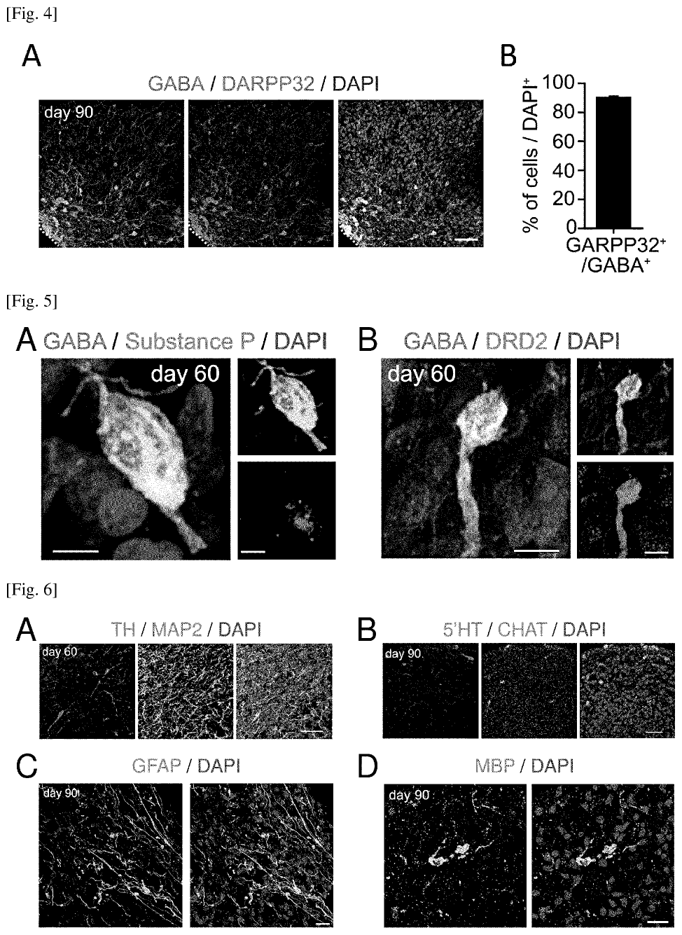

Here, by labeling our neurons in hSLOs with CTIP2 and GABA, we conclude that there are 64% of GABAergic neurons are also positive with CTIP2 day 60 (FIG. 3). Previous studies have indicated that the homeobox gene GSX2 is important to determine the LGE fate and striatum development (Hsich-Li et al., 1995; Toresson et al., 2000; Yun et al., 2001). By labeling day 30 hSLOs cryosections with GSX2 and a ventral forebrain marker DLX2, we observed a majority of GSX2+ cells also express DLX2 (83%). Further characterization of the mature hSLOs at day 60 showed that COUP-TF-interacting protein 2 (CTIP2), which are striatal markers, expressed in GABA⁺ neurons. Lastly, immunostaining of hSLOs cryosections at day 90 confirmed the expression of DARPP32, a marker of mature MSNs with 90% cells co-expressed DARPP32⁺/GABA⁺ (FIG. 4). More interestingly, immunostaining of hSLOs neurons with Subtance-P and DRD2 antibodies identified a certain population of GABA⁺ neurons express these two markers individually (FIG. 5). This feature is indeed very important as currently, there is no such 2D or 3D human striatal culture demonstrating the generation of human striatum with high percentage of D1 and D2 GABAergic neurons.

Different studies concluded that besides MSNs, human striatum contains a small population of supporting cells to promote neuronal survival as well as to maintain synaptic formation (Huot et al., 2007). We found that cells in our hSLO express TH (dopaminergic neuron makers), CHAT and 5-HT (Cholinergic neurons and serotonin neuron markers), MBP (oligodendrocyte markers) as well as GFAP (astrocyte markers) (FIG. 6).

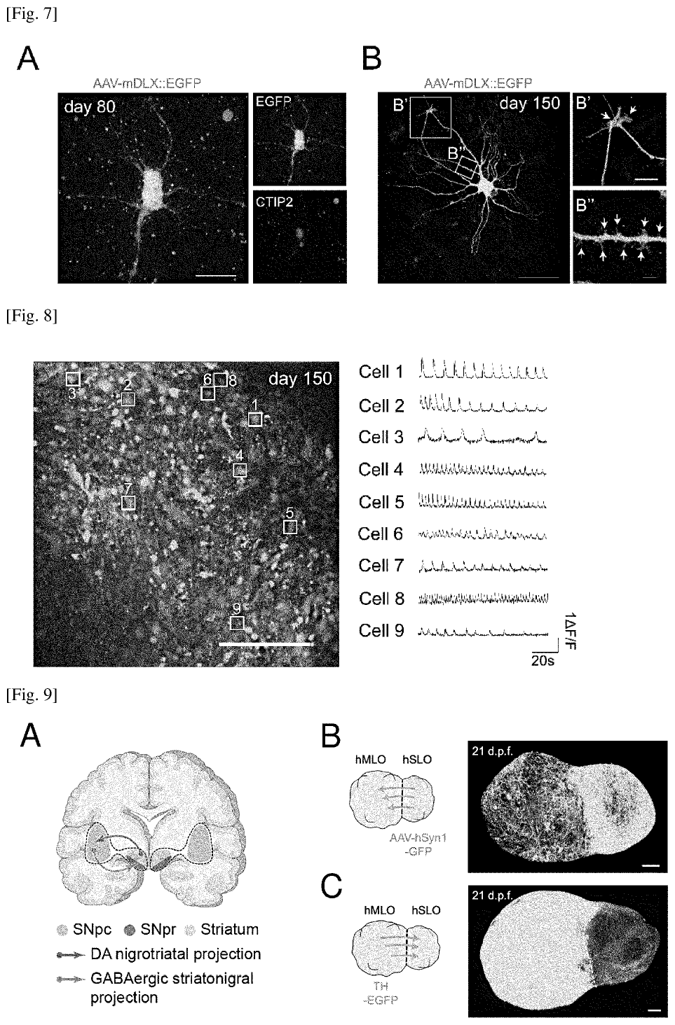

To further characterize the morphology of MSN including dendritic spines and growth cones as projection neurons, we labeled organoids with an AAV virus expressing EGFP reporter under DLX5 and DLX6 enhancer (AAV-mDlx::EGFP) and replated them to promote axonal outgrowth of MSNs. As a result, we observed a population of EGFP⁺ cell that are labeled with CTIP2 markers at day 80 (FIG. 7A). By further culturing neurons up to 130-150 days, we found that most EGFP⁺ neurons grew into a more complex morphology with many axonal branches. More interestingly, we captured growth cones at the axon terminus as well as the formation of dendritic spines (FIG. 7B), showing that the neurons became mature and were active to form synapses with other neurons.

Calcium imaging allows us to monitor the electrophysiological activity of individual neurons in brain organoids. For that purpose, we performed Fluo-4 acetoxymethyl ester (AM)-based calcium imaging. Incubating hSLOs with Fluo-4 AM resulted in labeled cells with prominent spontaneous Ca²⁺ transients. The recorded activity was analyzed using ImageJ software showed the spiking of single neurons of day 150 hSLOs (FIG. 8). Since the calcium activity of hSLOs can be measured using confocal microscope, measuring calcium imaging of fusion organoids can be a promising assay that allowed directly accessing the function of fusion organoid in PD models.

All these results suggest that the hSLOs we generated can produce MSN GABAergic neurons with multiple features that resemble to the human striatum. More importantly, we are analyzing bulk and single cell RNA sequencing data from hSLOs to further understand the transcriptomic characterization and the composition of cell types in hSLOs, as well as to compare the neurons in hSLOs with those derived from the previous published protocols.

### Example 2

Fusion of hSLOs and hMLOs Recapitulates Reciprocal Projections

We expected that the projected neurons in hMLO-hSLO fusion organoids can form a better functional synaptic connection and hence, we aim to record more active Ca²⁺ activity in fusion organoids. Reciprocal projections between midbrain substantia nigra and striatum where is a specific region of basal ganglia arise during neural development (FIG. 9A). To efficiently visualize neural projections, we built a reporter system in hMLOs and hSLOs. We utilized CRISPR/Cas9 technology to knock-in EGFP fluorescence protein into 3′-TH locus, in which specifically expresses in DA neurons of hMLOs. On the other hand, we utilized AAV infection system carrying hSyn1::EGFP (AAV-hSyn1::EGFP) to label GABAergic MSNs in hSLOs. To establish these projections in vitro we placed hSLOs and hMLOs next to each other in 24 well plate to promote their direct physical contact to fuse them together. Two days after fusion, organoids were placed back into orbital shaker to further culture for 2 weeks.

To demonstrate the striato-nigral projection, we fused hSyn1::EGFP infected hSLOs with no-labeled hMLOs. Interestingly, from 3 days post fusion we started to observe the projection. Two weeks after fusion, we found a robust extention of EGFP⁺ processes from hSLO reaching the opposite side of hMLOs and forming axonal bundles (FIG. 9B), which is similar anatomical feature of human brain (Morello et al., 2015). Similarly, to examine the projection capacity from hMLOs to hSLOs, we then fused TH-EGFP reporter hMLOs with no-labeled hSLOs. As expected, we detected EGFP⁺ processes to hSLOs side in 2 weeks post fusion (FIG. 9C). With longer culture, we have seen an augmented intensity of EGFP labeled projection signals from both fusion organoids. Together, these results indicated that there are reciprocal projections between hSLOs and hMLOs, suggesting that the fusion organoids are anatomical replicas of human neural circuit.

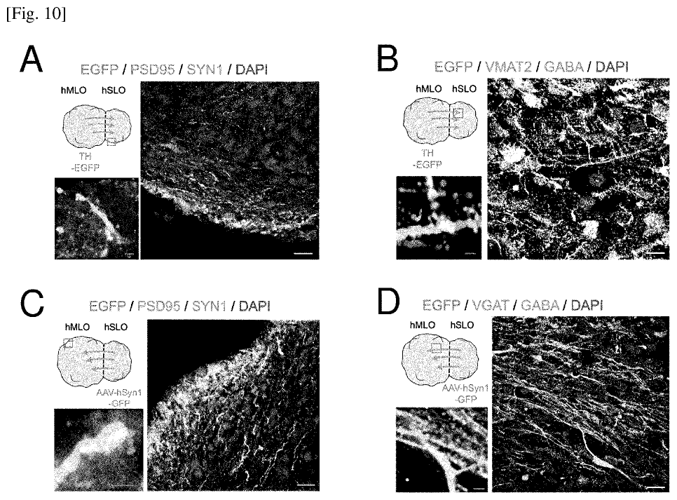

We further performed a series of assays to investigate the synaptic properties of fusion organoids. First, to determine whether projected neurons can direct axonal targeting and synaptogenesis in the fusion organoids, we fused TH-EGFP reporter hMLOs with no-labeled hSLO, we then labeled projected neurons together with pre-synaptic markers at the projected axonal terminal to convince the establishment of synaptic connectivity in the projected neuron. As expected, we observe the expression of SYN1 pre-synaptic marker and PSD95 post-synaptic marker along with the THEGFP⁺ projected neurons on hSLO side. Moreover, we observed the expression of vesicular monoamine transporter 2 (VMAT2) along with the TH-EGFP⁺ projected neurons and local GABAergic neurons (FIG. 10). In addition, we observe the expression of SYN1 pre-synaptic marker and PSD95 post-synaptic marker along with the AAV-hSyn1::EGFP⁺ projected neurons on hMLO side. Moreover, we observed the expression of vesicular GABA transporter (VGAT) along with the AAV-hSyn1::EGFP⁺ projected neurons and local GABAergic neurons, demonstrating the synaptic formation of projected DA neurons and GABAergic neurons with local GABAergic neurons.

Next, electrophysiological recording including calcium imaging, MEA, and whole cell patch clamp can be used to investigate the functional maturation and formation of neuronal circuit in fusion organoids. We expected that projected neurons can form better synapses with their counterparts when fusing, which is missing in each organoid alone.

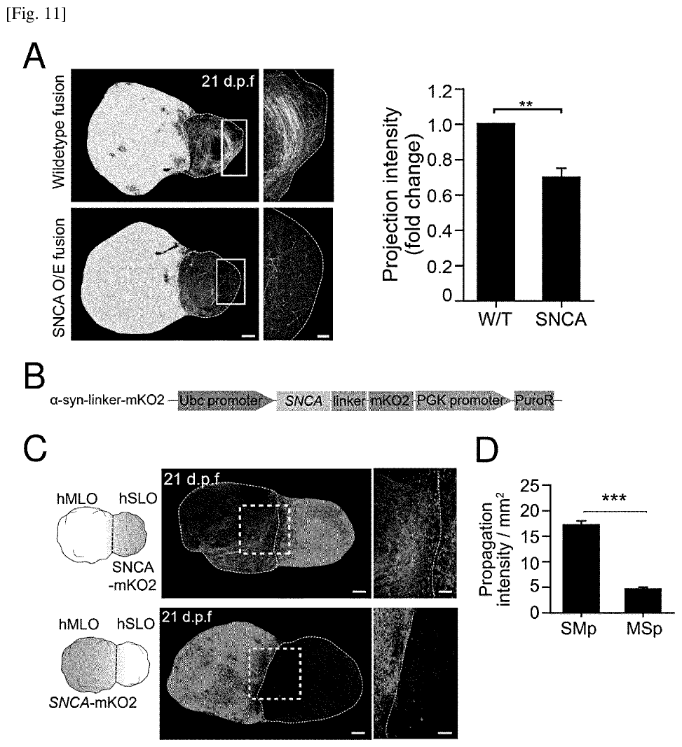

To demonstrate the possibility of using our fusion organoid system to model the asyn pathologies, we compare hMLO-hSLO fusion organoids between WT and SNCA overexpression (FIG. 11A). By labeling TH⁺ DA neurons in hMLOs, we demonstrated that the elevated α-Syn expression limits the projection of DA neurons from hMLOs to hSLOs. Interestingly, WT DA neurons projected as bundle whereas DA neurons from SNCA overexpression organoids showed fewer and random projection We next utilize lentivirus system to overexpress mKO2 reporter (red fluorescent protein) fused with α-syn (FIGS. 11B and C). This reporter system allowed us to visualize the aggregation of α-syn as well as monitor the action of α-syn propagation in hMLO-hSLO fusion organoids. By doing whole mount staining using mKO2 antibodies, we observe significant higher in the propagation level of α-Syn-mKO2 from hSLOs to hMLOs, but not vice versa, with almost 4 folds difference (FIG. 11C-D). These data strongly suggests that SNCA-mKO2 reporter is a feasible system to investigate α-syn propagation and the formation of α-syn aggregation between striatum and midbrain substantia nigra using our hMLO-hSLO fusion organoid model.

### Example 3

### The Application of Using Fusion Organoid for Drug Screening and Test for Synucleinopathy

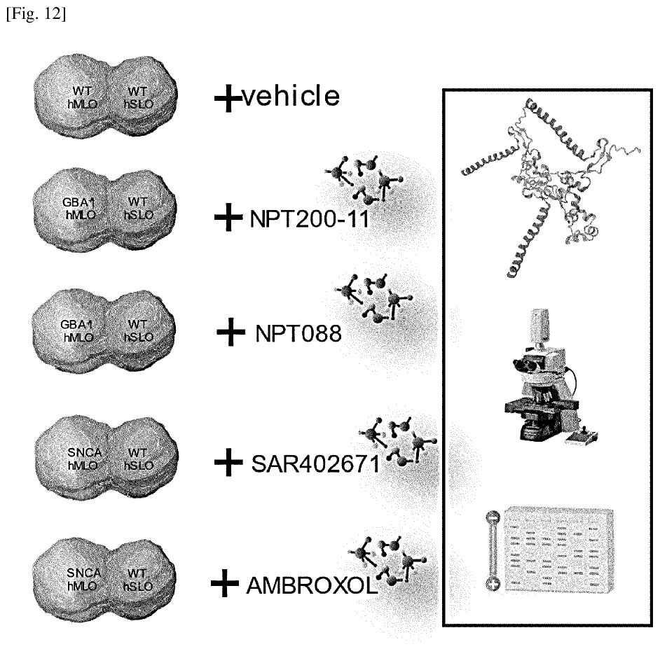

The main aim of utilizing hMLO-hSLO fusion organoid model is to develop a system that revolutionize to study pathogenesis of PD in reciprocal projections between midbrain substantia nigra and striatum, particularly focusing on synuclein pathology. We aim to test the effect and impact of anti-PD drug compounds in clinical trials using the fusion organoid in vitro system. Utilizing the isogenic PD hESC lines (Jo et al., 2021) with TH-EGFP reporter system that have been established in our lab, we generate fusion organoids demonstrating PD phenotypes (FIG. 12). First, we test a drug (for example, a compound, nucleic acid, or antibody) or candidate drugs in Phase I clinical trial (NPT200-11 and NPT088: inhibitor of α-syn misfolding) and Phase II clinical trial (SAR402671: inhibitor of glucosylceramide synthase, and Ambroxol: GCase activator) for their effect to rescue α-syn pathology. After that, we measure the degree of α-syn aggregation at the different time points exhibiting different extent of the PD phenotype and apply the drugs with optimized concentration to profile long-term and direct drug response on the fusion organoids. The assessment of the ameliorated level of pathological α-syn in TH+DA neurons will be validated by various approaches including multi-omics analyses, imaging, biochemistry, electrical properties investigation, metabolic assays, and the capacity to form synaptic formation both in physical and functional manners. We expect the outcome to decrease the accumulation of α-syn associated with reduced neurodegeneration following the drug treatments.

Parkinson's disease (PD) is the second most common neurodegenerative disorder, demonstrated by the degeneration of dopaminergic (DA) neurons, which normally project from midbrain to striatum in nigro-striatal pathway. Building an in vitro system to model neuronal diseases is challenging but is an achievable goal that many research groups attempt. Here, we developed a detailed protocol to produce specific human striatal-like organoid (hSLOs) with features similar to human striatum, such as the presence of D1 and D2 subtypes GABAergic medium spiny neurons (MSNs). By fusing hSLOs with our previously generated midbrain-like organoids (hMLOs), we provide in vitro evidence of connection and communication between midbrain and striatum in basal ganglia. Finally, we provide evidence that our fusion organoid system is a suitable drug screening platform against Parkinson's disease based on α-synuclein (α-syn) propagation. This finding represents the first attempt that can revolutionize in vitro neurodegenerative disease modeling, especially synucleinopathy, based on looking at both structural and functional interaction between hSLOs and hMLOs.

Each of the references and patents and patent applications cited herein has been incorporated by reference in its entirety.

### REFERENCES

Albin, R. L., Young, A. B., and Penney, J. B. (1989). The functional anatomy of basal ganglia disorders. Trends Neurosci 12, 366-375.
Arber, C., Precious, S. V., Cambray, S., Risner-Janiczek, J. R., Kelly, C., Noakes, Z., Fjodorova, M., Heuer, A., Ungless, M. A., Rodriguez, T. A., et al. (2015). Activin A directs striatal projection neuron differentiation of human pluripotent stem cells. Development 142, 1375-1386.
Gerfen, C. R. (2006). Indirect-pathway neurons lose their spines in Parkinson disease. Nat Neurosci 9, 157-158.
Goto, S., Hirano, A., and Matsumoto, S. (1989). Subdivisional involvement of nigro-striatal loop in idiopathic Parkinson's disease and striatonigral degeneration. Ann Neurol 26, 766-770.
Graveland, G. A., and DiFiglia, M. (1985). The frequency and distribution of medium-sized neurons with indented nuclei in the primate and rodent neostriatum. Brain Res 327, 307-311.
Hikosaka, O., Takikawa, Y., and Kawagoc, R. (2000). Role of the basal ganglia in the control of purposive saccadic eye movements. Physiol Rev 80, 953-978.
Hsich-Li, H. M., Witte, D. P., Szucsik, J. C., Weinstein, M., Li, H., and Potter, S. S. (1995). Gsh-2, a murine homeobox gene expressed in the developing brain. Mech Dev 50, 177-186.
Huot, P., Levesque, M., and Parent, A. (2007). The fate of striatal dopaminergic neurons in Parkinson's disease and Huntington's chorea. Brain 130, 222-232.
Ingham, C. A., Hood, S. H., Taggart, P., and Arbuthnott, G. W. (1998). Plasticity of synapses in the rat neostriatum after unilateral lesion of the nigrostriatal dopaminergic pathway. J Neurosci 18, 4732-4743.
Jo, J., Xiao, Y., Sun, A. X., Cukuroglu, E., Tran, H. D., Goke, J., Tan, Z. Y., Saw, T. Y., Tan, C. P., Lokman, H., et al. (2016). Midbrain-like Organoids from Human Pluripotent Stem Cells Contain Functional Dopaminergic and Neuromelanin-Producing Neurons. Cell stem cell 19, 248-257.
Jo, J., Yang, L., Tran, H. D., Yu, W., Sun, A. X., Chang, Y. Y., Jung, B. C., Lee, S. J., Saw, T. Y., Xiao, B., et al. (2021). Lewy Body-like Inclusions in Human Midbrain Organoids Carrying Glucocerebrosidase and alpha-Synuclein Mutations. Ann Neurol.
Lovinger, D. M. (2010). Neurotransmitter roles in synaptic modulation, plasticity and learning in the dorsal striatum. Neuropharmacology 58, 951-961.
Macpherson, T., Morita, M., and Hikida, T. (2014). Striatal direct and indirect pathways control decision-making behavior. Front Psychol 5, 1301.
Morello, F., Prasad, A. A., Rehberg, K., Vieira de Sa, R., Anton-Bolanos, N., LeyvaDiaz, E., Adolfs, Y., Tissir, F., Lopez-Bendito, G., and Pasterkamp, R. J. (2015).
Frizzled3 Controls Axonal Polarity and Intermediate Target Entry during Striatal Pathway Development. J Neurosci 35, 14205-14219.
Toresson, H., Potter, S. S., and Campbell, K. (2000). Genetic control of dorsal-ventral identity in the telencephalon: opposing roles for Pax6 and Gsh2. Development 127, 4361-4371.
Yager, L. M., Garcia, A. F., Wunsch, A. M., and Ferguson, S. M. (2015). The ins and outs of the striatum: role in drug addiction. Neuroscience 301, 529-541.
Yun, K., Potter, S., and Rubenstein, J. L. (2001). Gsh2 and Pax6 play complementary roles in dorsoventral patterning of the mammalian telencephalon. Development 128, 193-205.

1. A method of culturing an embryoid body (EB) such as a human embryoid body, comprising:
culturing an EB in a first culture medium containing a first factor, wherein the first factor comprises a TGF-β signaling pathway inhibitor and/or Wnt inhibitor such as GSK inhibitor.

2. The method of claim 1, further comprising:
culturing the obtained EB in a second culture medium containing a second factor, wherein the second factor comprises a Wnt inhibitor, patterning factors for sonic hedgehog pathway activation, and activin without first factors to obtain an LGE neurospheres.

3. The method of claim 2, further comprising:
culturing the obtained EB in a third culture medium containing a third factor, wherein the third factor comprises brain-derived neurotrophic factor (BDNF) and/or ascorbic acid without the first and second factors to obtain an organoid such as a human striatal-like organoid.

4. The method of claim 3, wherein the obtained organoid comprising one or more markers of a mature medium spiny neuron (MSN).

5. The method of claim 4, wherein the obtained organoid comprising D1 and/or D2 MSN.

6. The method of claim 5, wherein the obtained organoid expresses at least one or all of interneuron makers such as TH, cholinergic neuron and serotonin neuron markers such as CHAT and 5-HT, glial cell markers such as MBP, and GFAP.

7. The method of any one of claims 4 to 6, wherein the organoid has 1 mm or more in major axis diameter or in diameter, preferably 1 mm to 2 mm in major axis diameter or in diameter.

8. An isolated organoid comprising: a mature medium spiny neuron (MSN) expressing one or more markers of a mature MSN.

9. The isolated organoid of claim 8, wherein 50% or more of the cells contained in the organoid are DARPP32 positive and GABA positive.

10. The isolated organoid of claim 8 or 9, comprising D1 and/or D2 MSN.

11. The isolated organoid of any one of claims 8 to 10, wherein the organoid expresses at least one or all of interneuron makers, cholinergic neuron markers, and serotonin neuron markers.

12. The isolated organoid of any one of claims 8 to 11, wherein the organoid has 1 mm or more in major axis diameter or in diameter.

13. A method of producing an organoid, comprising:
providing an organoid of any one of claims 8 to 12 (a first organoid), and a second organoid comprising a dopaminergic (DA) neuron, and contacting the first organoid and the second organoid to obtain a fusion organoid of the first organoid and the second organoid, wherein a dopaminergic neuron in the second organoid has projection that reaches the first organoid, and a GABAergic MSN in the first organoid has projection that reaches the second organoid.

14. A fusion organoid of the first organoid of any one of claims 8 to 12 and the second organoid comprising a dopaminergic (DA) neuron, wherein a dopaminergic neuron in the second organoid has projection that reaches the first organoid, and a GABAergic MSN in the first organoid has projection that reaches the second organoid.

15. The fusion organoid of claim 14, wherein the cells included in the fusion organoid comprises a DA neuron having α-syn aggregation.

16. A method of testing a candidate drug, comprising:
contacting the candidate drug and the fusion organoid of claim 14 or 15,
observing α-synuclein aggregation in a DA neuron, and
selecting the candidate drug that decreases α-syn aggregation, compared to a negative control.

17. The method of claim 16, wherein SNCA is overexpressed in at least a DA neuron or all cells in the second organoid entity or the organoid.
